(19) United States
(12) Patent Application Publication
(10) Pub. No.: US 2025/0329040 A1
(43) Pub. Date: Oct. 23, 2025

(54) METHOD AND APPARATUS FOR SUPPORTING A DETERMINATION OF A VESSEL MORPHOLOGY ON THE BASIS OF SPECTRAL CT INFORMATION

(71) Applicant: Siemens Healthineers AG, Forchheim (DE)

(72) Inventors: Thomas ALLMENDINGER, Forchheim (DE); Thomas FLOHR, Uehlfeld (DE); Bernhard SCHMIDT, Fuerth (DE)

(73) Assignee: Siemens Healthineers AG, Forchheim (DE)

(21) Appl. No.: 19/180,274

(22) Filed: Apr. 16, 2025

(30) Foreign Application Priority Data
Apr. 18, 2024 (DE) 10 2024 203 610.2

(51) Int. Cl. G06T 7/62; A61B 6/00; A61B 6/03; G06T 7/00; G06T 7/10

(52) U.S. Cl. CPC G06T 7/62; A61B 6/03; A61B 6/5205; G06T 7/0012; G06T 7/10; G06T 2207/10081; G06T 2207/20092; G06T 2207/30101

(57) ABSTRACT

One or more example embodiments relates to a method for supporting a determination of a vessel morphology on the basis of spectral CT information. Furthermore, one or more example embodiments comprises an apparatus, a control facility and a medical technology system.

### CROSS-REFERENCE TO RELATED APPLICATION(S)

The present application claims priority under 35 U.S.C. § 119 to German Patent Application No. 10 2024 203 610.2, filed Apr. 18, 2024, the entire contents of which is incorporated herein by reference.

### Field

One or more example embodiments relates to a method and an apparatus for supporting a determination of a vessel morphology on the basis of spectral CT information, a control facility for a medical technology system and a medical technology system.

### Related Art

The generation of CT images of the vessels typically takes place in the context of a recording with iodine contrast medium and a subsequent reconstruction of the images as “standard HU” images (HU: Hounsfield units). Difficulties can often arise in the evaluation of the images if, for example, calcifications occur in the vessel and, as a result, the calcification is displayed enlarged due to its high intrinsic contrast level and thus overexposes the visible lumen.

These partial volume effects are increasingly gaining importance in the context of computer-assisted evaluation since they necessitate an exact quantitative evaluation of the morphology of the individual regions and constituents of a vessel for further processing (contours or surface networks). Apart from the active vessel diameter (lumen as a percentage or in millimeters), what is concerned here is the quantitative determination of plaque volumes, e.g. in cubic millimeters, which are typically still classified into the groups soft, fatty and hard plaques.

In the prior art there is a series of methods for determining the relevant vessel diameter, plaque sizes and stenosis level. Typically, the following steps are required: determining the center of the vessel with subsequent calculation of line profiles orthogonal thereto from different directions. Apart from the simplest evaluation of the profiles with the aid of fixed HU thresholds, the first and second derivatives of the HU profiles are also often used, which offers a more robust measure of the geometric object sizes through evaluation of the local maxima or minima or zero-crossings, for example, by way of a check of the influence of the point spread function of the reconstruction kernel used or an implicit derivation of the full width half maximum through the zero-crossings of the second derivative.

### SUMMARY

However, according to the current prior art, the methods are restricted in their practical application to “standard HU” images, the evaluation of which can lead to serious errors.

One or more example embodiments provides a method and an apparatus for supporting a determination of a vessel morphology on the basis of spectral CT information, a control facility for a medical technology system and a medical technology system with which the disadvantages described above are avoided.

This is achieved by way of a method according to claim 1, an apparatus according to claim 10, a control facility according to claim 12 and a medical technology system according to claim 13.

### BRIEF DESCRIPTION OF THE DRAWINGS

At least some example embodiments will now be described again in greater detail, making reference to the accompanying drawings. In the various drawings, the same components are provided with identical reference signs. The drawings are, in general, not to scale. In the drawings:

FIG. 1 shows a rough schematic representation of a CT system with an exemplary embodiment of a control facility according to one or more example embodiments for carrying out the method,

FIG. 2 shows a block diagram of the sequence of the method,

FIG. 3 shows a detection of a stenosis, and

FIG. 4 shows absorptions by different contrast media.

### DETAILED DESCRIPTION

A method according to one or more example embodiments serves for supporting a determination of a vessel morphology on the basis of spectral CT information. It comprises the following steps:
providing at least two 3D CT images of a person which have been recorded with different recording energies, wherein at least one low energy image and a high energy image are provided,
selecting a vessel of the person and an examination position at which the vessel is to be investigated,
generating an examination image by superimposing image information of at least the low energy image and the high energy image, wherein the examination image shows a cross-section through the selected vessel at the examination position in a view from above,
determining a checking region in the cross-section and forming a working set from image values of this checking region,
calculating at least the first derivative of the working set with respect to the location, and
establishing a number of change regions in the progression of the image values of the working set on the basis of the first derivative,
outputting information regarding positions of the number of change regions.

The two 3D CT images can be provided in the form of corresponding image datasets.

For the method, two or more CT images of a person must be available. These CT images show the same region of the person. Spectral CT images are often recorded simultaneously and each show the same subject. However, this is not absolutely essential for the method. The CT images can certainly have been recorded one after the other. They can also show different image portions, provided both show the region of interest (ROI).

The CT images must be recorded, but it is not necessary for them to be used by the method immediately following the recording. They can also be stored in a memory store after the recording, and thereafter loaded again from this memory store, so that following their recording and the processing by the method, some time can certainly elapse.

As usual for a recording of vessels, the CT images should have been recorded after the administration of a contrast medium. The recording of spectral images following the administration of a contrast medium is known from the prior art. Therein typically, images are recorded of which the recording energy of one is in the region of a high absorption by the contrast medium, in particular, in the region of its k-edge and the recording energy of the other is in a region in which the substance to be investigated has a higher absorption than the contrast medium. In principle, recording energies should be selected at which the contrast medium and the substance to be investigated can be well separated from one another. Even though iodine is an often preferred contrast medium, other contrast media or two or more contrast media (with different k-edges) can be used. Therein, to distinguish between contrast media, more than two recording energies can also be used.

Therein, at least two CT images can be recorded with different recording energies. That with the lowest recording energy is designated the “low energy image” and that with the highest is designated the “high energy image”. In addition, further images can be used, for example, DER (dual-energy ratio) images, PureLumen (an image in which the calcium content has been calculated by using the spectral information) or a plurality of intermediate energies.

It should be noted that the method according to one or more example embodiments relates to an automated processing of images. Thereby, the representation of these images can be improved for a later diagnosis performed by a person, but it does not perform a diagnosis itself.

For the method, a vessel in the person (in the ROI) must be sought, and also an examination position (also in the ROI) at which the vessel is to be investigated. A plurality of vessels can also be sought, although this is, in principle, equivalent to multiple executions of the method. The vessels can be sought following the recording of the CT images, during it or before it.

The vessel can be sought automatically, preferably in that a segmentation of the image is performed, vessels are identified and then the method is carried out for a number, for example all, of the segmented vessels. A vessel can however also be sought manually by a user. It is also conceivable that an automatic selection of all the vessels is carried out in an ROI and a user manually selects at least one of the automatically selected vessels, for which the method is applied. However, the selection of a vessel can also be made dependent upon a presetting or a preselection for a vessel of interest and can take place automatically on the basis of the presetting or preselection.

If the CT images are available and if the examination position is known, then the examination image is generated. It is created by superimposing image information, of at least the low energy image and the high energy image, preferably by way of a subtraction or division of the image values at the respective same position from one another. However, other forms of the generation of the examination image are conceivable, for example, weighted or unweighted addition, subtraction, multiplication or division of image values or more complex superimpositions, for example, for generating a virtual mono-energetic image or for generating a material-specific image with the aid of a material decomposition.

The respective same position is intended to mean the absolute position of the ROI. Essentially, an identical ROI is sought in at least the CT images, these regions of the CT images are suitably overlaid on one another and image values lying over one another are overlaid so that a superimposed image of the ROI results. The examination image is thus formed from image points, the image values of which each represent a superimposition of mutually corresponding image points of the CT images.

The examination image shows a cross-section through the selected vessel at the examination position in a view from above. This means that it shows the view into the vessel. Although it is preferred that the cross-section is made orthogonally to the extent of the vessel, in practice oblique cross-sections are also possible. It is sufficient for a simple execution of the method if the examination image is a two-dimensional image. However, the examination image can also be three-dimensional, for example, if a spatially extended region is to be investigated.

In the cross-section, that is, in the examination image, a checking region is then determined. This checking region discloses which image values of the examination image are to be further processed. The checking region can be a line, a plurality of lines, for example, a cross, or an area, for example, the whole cross-section.

The checking region can be determined automatically, preferably in that a predetermined type (e.g. a line or an area) is selected for a checking region and is applied to the cross-section. However, the checking region can also be determined by a user, preferably by way of manually marking a region.

Once the checking region has been determined, a working set of image values of this checking region is formed. If the checking region is a line, then the working set is formed from the image values of image points (pixels/voxels) along this line and if it is an area, then the working set is formed from the image values of image points (pixels/voxels) on this area. Since derivatives are subsequently formed, the elements of the working set should have information relating to their positions in addition to the image value. Since pixels or voxels meet this requirement, it is preferable that the working set is formed from pixels or voxels. Thus, the pixels or voxels of the examination image in the checking region can simply be copied and included in the working set.

Once the working set has been formed, the first derivative of the working set can be formed with respect to the location. If the checking region was a line, then the working set essentially forms a graph of image values (Y-coordinates) along this line (X-coordinates). This graph can then be derived with respect to X, the location. Use can therein be made of the fact that the elements of the working set (pixels/voxels) have fixed spacings from one another. For the first derivative, from respective adjacent elements Ei, Ej essentially only the difference Ej-Ei needs to be formed.

If the checking region was two-dimensional, then the working set represents an area in an X-Y plane with image values in a Z direction, then it can be considered to be a scalar field and as the first derivative, partial derivatives are calculated in the X and Y directions. The result would be a vector field in which two values per image point specify the derivative with respect to X and Y, respectively.

If the checking region was three-dimensional, the working set can represent a volume in an X-Y-Z space with image values in a further dimension. Then, as the first derivative, partial derivatives in the X, Y and Z directions can be calculated. The result would be a multidimensional vector field.

Following the formation of the first derivative, a second derivative can also be formed. This can support the subsequent establishment.

After the derivation, the progress of the image values can be observed with respect to a change. Preferably, extreme points (local maxima and minima) of the first derivative are considered which would be expressed as zero points of a second derivative. Therein, a change region is always situated between two extreme points since this means that the image values are systematically different there from beyond these extreme points. In an optimum vessel, for example, two extreme sites can be expected (if the checking region was a line), specifically on the walls of the vessel. By way, at least, of a previously applied contrast medium, the image values in the interior of a vessel should differ distinctly from the region outside the vessel. If the vessel has a plaque, then there could be, for example, three extreme sites: one at each edge of the vessel and one in the interior at the edge of the plaque. There would thus be two change regions in the interior of the vessel. Through the special procedure, specifically the formation of the examination image by way of the superimposition of image values, the derivatives become much more meaningful since the transitions in the examination image are clearer than in normal HU images.

The information, for example, positions and possibly mean image values, over this number of change regions can then be output, for example, as an image or as numerical values and can assist a person with his diagnosis.

With the aid of a use of spectral CT images from corresponding recordings, for example, dual energy or photon counting CT (PCCT), one or more example embodiments therefore enables an optimized determination of morphological variables of a vessel region, for example, lengths, diameters, relative lengths, volumes or a stenosis level. In particular, the one or more example embodiments enables the diameter and the stenosis level of vessels with plaque (soft, fatty, calcified, . . . ) to be determined better and more robustly. The vessel profiles derived from this information and its derivatives show structures either more clearly or for the first time, in comparison with classic profile-based methods which use standard HU images. This has the advantage that, in particular, diameters and volumes can be determined with greater accuracy and smaller errors.

An apparatus according to one or more example embodiments serves for supporting a determination of a vessel morphology on the basis of spectral CT information. It comprises the following components:
a data interface configured for receiving image data from 3D CT images of a person, that have been recorded with different recording energies, at least from a low energy image and a high energy image.
a selecting unit configured for selecting a vessel of the person and an examination position at which the vessel is to be investigated,
a superimposing unit configured for generating an examination image by superimposing image information of at least the low energy image and the high energy image, wherein the examination image shows a cross-section through the selected vessel at the examination position in a view from above,
a working set unit configured for determining a checking region in the cross-section and forming a working set from image values of this checking region,
a calculating unit configured for calculating at least the first derivative of the working set with respect to location,
a change unit configured for establishing a number of change regions in the progression of the image values of the working set on the basis of this derivative,
a data interface configured for outputting positions of the number of change regions.

The function of the components of the apparatus has already been described above. The apparatus is preferably configured to carry out a method according to one or more example embodiments.

Preferably, the selecting unit is configured to search automatically for a vessel, preferably in that it is configured to carry out a segmentation of the image. Alternatively or additionally it can, however, also make a user interface available for a manual selection of a vessel by a user.

Preferably, the working set unit is configured to determine a checking region automatically, preferably in that a predetermined type (e.g. a line or an area) is selected for a checking region and is applied to the cross-section. Alternatively or additionally it can, however, also make a user interface available for a manual determination of a checking region, in particular, with which a user can mark a checking region manually.

A control facility according to one or more example embodiments for a medical technology system, preferably a CT system or a diagnostic system, comprises an apparatus according to one or more example embodiments and/or is configured to carry out a method according to one or more example embodiments.

A medical technology system according to one or more example embodiments, preferably a diagnostic system or a CT system, in particular, a photon counting CT system, comprises a control facility according to one or more example embodiments.

In particular, the features and advantages described in relation to the method according to the one or more example embodiments can also be configured as corresponding subunits of the apparatus according to one or more example embodiments or of the medical technology system according to one or more example embodiments. Conversely, the features and advantages described in relation to the apparatus according to one or more example embodiments or of the medical technology system according to one or more example embodiments can also be configured as corresponding method steps of the method according to one or more example embodiments.

One or more example embodiments can be realized, in particular, in the form of a computer unit with suitable software. For this purpose, the computer unit can have, for example, one or more cooperating microprocessors or suchlike. In particular, it can be realized in the form of suitable software program parts in the computer unit. A realization largely through software has the advantage that conventionally used computer units can also easily be upgraded with a software or firmware update in order to operate in the manner according to one or more example embodiments. The object is therefore also achieved, in particular, with a corresponding computer program product having a computer program which can be loaded directly into a memory storage facility of a computer unit, having program portions in order to carry out all the steps of the method according to one or more example embodiments when the program is executed in the computer unit. Such a computer program product can comprise, apart from the computer program, where relevant, additional constituents, such as, for example, documentation and/or additional components, and also hardware components, such as, for example, hardware keys (dongles, etc.) in order to use the software.

For transport to the computer unit and/or for storage at or in the computing unit, a computer-readable medium, for example a memory stick, a hard disk or another transportable or permanently installed data carrier can be used on which the program portions of the computer program which can be read in and executed by a computer unit are stored.

Further particularly advantageous embodiments and developments of one or more example embodiments are disclosed in the dependent claims and the following description, wherein the claims of one claim category can also be further developed similarly to the claims and description passages relating to another claim category and, in particular also, individual features of different exemplary embodiments or variants can be combined to new exemplary embodiments or variants.

Typically the CT images are formed from image elements, for example, pixels or voxels. The images then result from the image values of the image elements at discrete positions. This also applies to the examination image. In the formation of the examination image, a common ROI of all the images is always considered and this ROI should be identically mapped in all the images except for their specific image values (position, size, in 2D case viewing angle also) in order to avoid errors. Otherwise, an image registration could take place for adjustment. Preferably, all the CT images should show the same subject, but with different spectral image information.

Preferably, the examination image is calculated from a quotient and/or a difference of image values of corresponding image elements (pixels/voxels) of the low energy image and of the high energy image (and possibly of further image elements).

Depending upon the desired type of examination, the checking region can be individually configured. Particularly useful results are obtained with a linear, cruciform or areal checking region. The working set results directly from the checking region.

Preferably, the working set is therein formed from image values on a line transversely through the vessel cross-section, wherein the first derivative of the working set is a derivative over the progression of image values along the line.

Alternatively, the working set preferably comprises the image points of the area of the cross-section wherein the first derivative of the working set are the partial derivatives of the working set along two linear independent spatial directions transverse to the cross-section.

It is also preferable that the working set comprises the image points of the volume of the vessel from the cross-section and a predetermined stretch along the vessel course, wherein the first derivative of the working set are the partial derivatives of the working set along three linearly independent spatial directions.

Preferably, a change region is established from extreme sites of the image values of the working set, that is maxima and minima of the first derivative or zero points of the second derivative. Alternatively, a change region can be established from an amplitude of the first derivative (root of the sum of the squared partial derivatives). Since this essentially concerns the recognition of edges or transitions in space, in the three-dimensional case, a spatial derivative ∂/∂x, ∂/∂y, ∂/∂z could be applied across all 3 spatial dimensions and subsequent formation of the amplitude

$$\sqrt{{( \partial / \partial x )}^{2} + {( \partial / \partial y )}^{2} + {( \partial / \partial z )}^{2}}$$

Preferably, a second derivative is formed from the first derivative and change regions are preferably established from this second derivative or from the first and the second derivative. Preferably, for this purpose, zero points of the second derivative are established.

During the generation of the examination image, a normalization and/or a calibration and/or an adaptation and/or a mapping of values preferably additionally takes place. In particular, in this framework, a multiplication of all the image information by a predetermined factor or a predetermined location-dependent function over the examination image is preferred. This is advantageous in order to impart an optimum expressiveness to the examination image.

It is preferred that before the recording of the CT images of a person, said person is given a contrast medium, in particular iodine and the CT images are recorded, with this contrast medium, in a region to be investigated. A procedure of this type is known from the prior art. By way of example, for an investigation of the coronary arteries, typically after administration of iodine, firstly a wait takes place for a time until the contrast medium floods into these vessels and then the CT recordings are made.

Preferably, therefore, CT images are provided, having been recorded from a person after administration of a contrast medium, in particular an iodine-containing contrast medium. The CT images can comprise image datasets which have been recorded after introducing this contrast medium in a region of this person that is to be investigated.

According to a preferred embodiment of the method, the CT images are recorded in the context of a photon-counting CT process. This PCCT process provides an energy-resolved scan. Preferably, therein the examination image is generated by superimposing image information of a number of additional CT images in addition to the low energy image and the high energy image. Preferably therefore CT images are provided which have been recorded in the context of a photon counting CT method.

The photon-counting detector of a PCCT scanner can convert the incident X-ray quanta directly into an electrical signal. Since the signal strength correlates with the energy of the respectively acquired X-ray quantum, each acquired X-ray quantum can be assigned to an energy region. Therein, the energy regions that can be distinguished from one another by adapting the readout electronics of the photon counting detector are predetermined. This enables the selection of energy regions in which the spectral differentiation of different materials, for example, calcium, iodine, water is optimized.

It is therein preferred that before the recording of the CT images of a person, said person is given a plurality of contrast media having different absorption edges. Very heavy contrast media with a high k-edge, for example, gold or tungsten, are preferred. In this way, a differentiated investigation can take place. The recording energies should be selected so that a different contrast medium always has an absorption maximum in the CT images. Due to the high spatial and spectral resolution with PCCT, this recording method is very advantageous for this use.

It is preferred that, in one embodiment of the method, a spectral information item from image values of the working set is additionally established and a change region is established on the basis of this spectral information item. Preferably, for this purpose, a progression of image values from different CT images is generated dependent upon the radiation energy and a derivative of the progression above a radiation energy is calculated.

Preferably, in order to determine a change region, the partial spectral derivative a/as (s being the spectral regions in which the CT images have been recorded) is also used for calculating a 4D edge amplitude. A direct inclusion of the spectral information item therein preferably takes place with formation of the amplitude \sqrt{(∂/∂x)²+(∂/∂y)²+(∂/∂z)²+(∂/∂s)²}. A corresponding second derivative can be calculated for three or more images, e.g. simply by subtraction of adjacent image values divided by the difference of the recording energies.

For this embodiment, the aforementioned PCCT (photon counting CT) method would also be very advantageous. This method can make CT images from a plurality of different spectral regions. Thus, spectral gradients d/ds (or ∂/∂s) can be formed and their significance acknowledged. A 4-D gradient could be formed with highly resolved spatial dimensions dx, dy, dz (for example, from the low energy image) in combination with the spectral derivative, wherein here images from other spectral regions would be considered which certainly also could use a lower spatial resolution. PCCT thus makes a mixed resolving capability available for this.

In a preferred apparatus, the superimposing unit is configured for superimposing three or more CT images to generate the examination image. The calculating unit is preferably additionally configured for calculating a first derivative of a spectral progression with respect to a radiation energy and the change unit is preferably configured to establish a change region on the basis of the derivative of the spectral progression.

The use of AI (artificial intelligence)-based methods for the method according to one or more example embodiments is preferable. An artificial intelligence system is based upon the principle of machine-based learning and is typically carried out with a learning-capable algorithm that has been suitably trained. For machine-based learning, the expression “machine learning” is often used, which here also includes the principle of “deep learning”. Such a use enables an optimum evaluation for determining the vessel size or plaque size on the basis of a plurality of different CT images on the basis of such a system to be trained and realized.

Preferably, components of one or more example embodiments are present as a “cloud service”. A cloud service of this type serves for processing data, in particular, via an artificial intelligence, but can also be a service on the basis of conventional algorithms or a service in which an evaluation by humans takes place in the background. In general, a cloud service (identified in the following as a “cloud”) is an IT infrastructure in which, for example, memory store space or computing power and/or an application software is made available via a network. The communication between the user and the cloud takes place via data interfaces and/or data transfer protocols. In the present case, it is particularly preferred that the cloud service makes both computing power and also application software available.

In the context of a preferred method, a provision of data that is obtained in the context of one or more example embodiments takes place via the network to the cloud service. This comprises a computer system which typically does not include the local computer of the user. The method can therein be realized via a command combination in a network. The data calculated in the cloud is preferably transferred again later via the network to the local computer of the user.

Preferably, as the low energy image and as the high energy image (and the further image) for the method, direct HU images can be used (HU=Hounsfield units).

A dual energy ratio (or an examination image) can be generated as a ratio of measured and identically reconstructed Qr40 low energy images and high energy images. A low energy image is therein divided by a corresponding high energy image. This can take place in that the image values of identical image elements are divided correspondingly.

For the examination image, it is preferred that a threshold is specified, in particular, on calculation of a quotient of image values. If HU images are provided, a threshold below 50 HU, in particular below 25 HU is preferred.

For example, a threshold can amount to 20 HU. Below this threshold, a predetermined value is preferably assigned to the image elements, for example, 1 HU.

The examination image can be normalized retrospectively. For example, in an examination image which results from a difference or a quotient of image values, its image values can be multiplied by a constant value. For example, the image values of an examination image that lie between 1 and 2.5 can be multiplied by 100 so that image values of between 100 and 250, that can be well represented as graduations of gray, result.

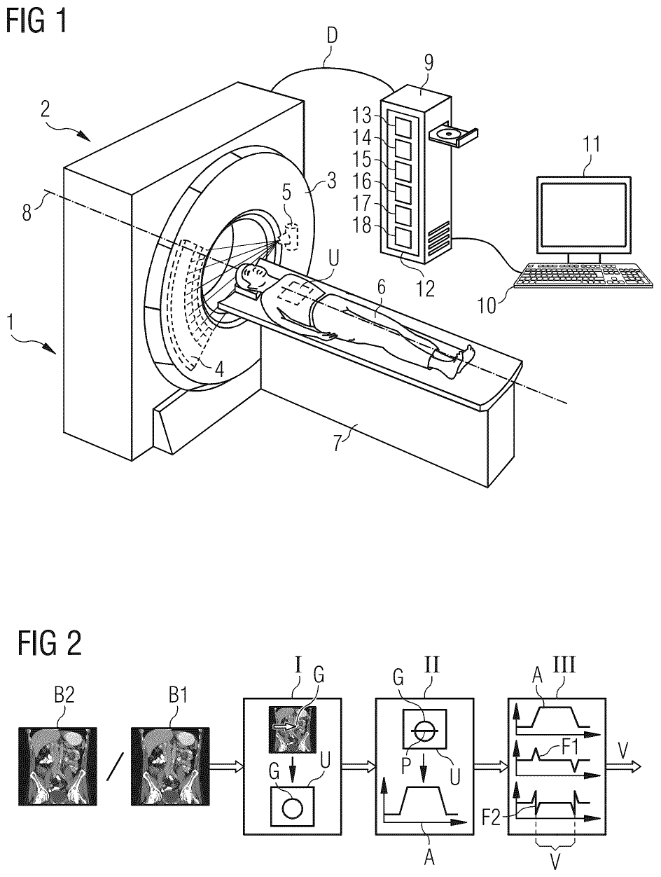

FIG. 1 shows an embodiment of a computed tomography (CT) system 1 with a radiation detector 4 and a radiation source 5. The radiation source 5 is configured to irradiate the radiation detector 4 with radiation. The CT system 1 shown comprises a gantry 2 with a rotor 3. The rotor 3 comprises, as the radiation source 5, an X-ray source 5, and the radiation detector 4 which is designed to detect X-ray radiation.

The rotor 3 is able to be rotated about the rotation axis 8. The patient 6 is positioned on the patient support 7 and is able to be moved along the rotation axis 8 through the gantry 2. The head of the patient 6 is supported on a positioning aid L. In order to control the imaging system 1 and/or to generate an image dataset on the basis of signals detected by the radiation detector 4, the computing unit 9 is provided.

Typically a (raw) X-ray image dataset of the examination object 6 is recorded from a large number of angular directions via the radiation detector 4 at one radiation energy, and therefore two or more raw datasets. Subsequently, on the basis of the (raw) X-ray image dataset, via a mathematical method, for example comprising a filtered back projection or an iterative reconstruction method, a (final) X-ray image dataset can be reconstructed.

The computing unit 9 serves here as a control facility 9 for controlling the CT system 1. An input facility 10 and an output facility 11 are connected to this computing unit 9. The input facility 10 and the output facility 11 can, for example, enable an interaction by a user or the representation of a generated image dataset B.

The control facility 9 comprises an apparatus 12 according to one or more example embodiments for supporting a determination of a vessel morphology on the basis of spectral CT information in accordance with a method according to one or more example embodiments (see FIG. 2). The apparatus 12 comprises a data interface 13, a selecting unit 14, a superimposing unit 15, a working set unit 16, a calculating unit 17 and a change unit 18.

The data interface 13 serves to receive image data of 3D CT images of a person, that have been recorded with different recording energies. At least a low energy image B1 and a high energy image B2 should be received.

In this example, the data interface 13 serves for outputting positions of the number of change regions V, that is of the result. These can be output, for example, on the output facility 11.

The selecting unit 14 serves for selecting a vessel G of the person and an examination position at which the vessel G is to be investigated. It can select a vessel G automatically, but it makes a user interface available via the input facility 10 and the output facility 11, via which interface a user can select a vessel.

The superimposing unit 15 serves for generating an examination image U by superimposing image information of at least the low energy image B1 and the high energy image B2, wherein the examination image U shows a cross-section through the selected vessel G at the examination position in a view from above.

The working set unit 16 serves for determining a checking region P in the cross-section and forming a working set A from image values of this checking region P. Herein, it can determine the checking region P automatically, but makes a user interface available via the input facility 10 and the output facility 11, via which a user can determine the checking region P.

The calculating unit 17 serves for calculating at least the first derivative of the working set A with respect to location.

The change unit 18 serves for establishing a number of change regions V in the progression of the image values of the working set A on the basis of this derivative.

FIG. 2 shows a block diagram of the sequence of a method for supporting a determination of a vessel morphology on the basis of spectral CT information.

Initially, two 3D CT images B1, B2 of a person are provided which have been recorded with different recording energies, wherein at least one low energy image B1 and one high energy image B2 are provided.

In step I, a selection of a vessel G of the person takes place and an examination position at which the vessel G is to be investigated, and an examination image U is generated by superimposing image information at least of the low energy image B1 and the high energy image B2. The examination image U shows a cross-section through the selected vessel G at the examination position in a view from above. In this example, the examination image U is a two-dimensional image.

In step II, a checking region P in the form of a line in the cross-section is determined and a working set A is formed from image values of this checking region P. This takes place simply in that the pixels of the examination image U along this line are defined as the working set A. The image values of the working set A along the line (checking region P) are shown here.

In step III, the first derivative F1 and the second derivative F2 of the working set A with respect to location are calculated and the change region V in the progression of the image values of the working set A is determined on the basis of the first derivative F1 and the second derivative F2.

The information determined in step III regarding positions of the change regions V is then output, for example, as a graphical representation that can be evaluated by a person.

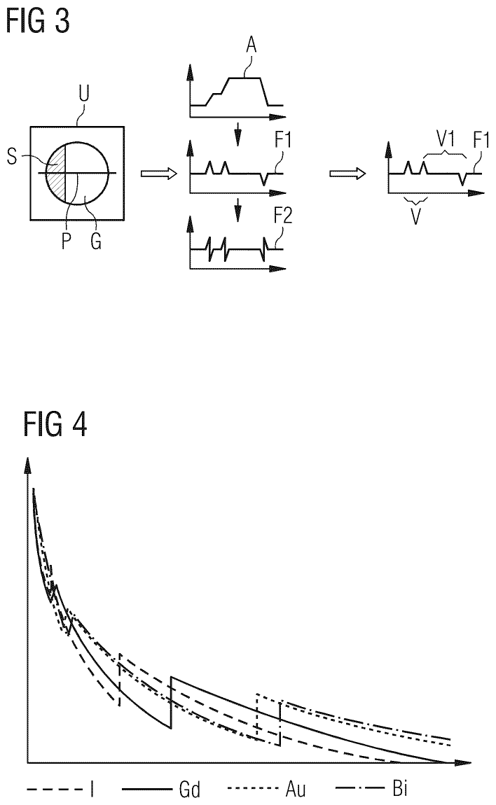

FIG. 3 outlines a detection of a stenosis according to the method as shown in FIG. 2. In the case shown here, the transition region P in the examination image U on the left is again a line since it relates to a very simple and readily representable case. The cross-section represented in the examination image through a vessel G has a stenosis S here which narrows the vessel G at the edge.

If the first derivative F1 and the second derivative F2 are now formed from the working set A, the transition from the edge of the vessel G to the stenosis S is discernible and also the transition to the clear region of the vessel and also from the clear region to the edge is very distinctly discernible in the derivatives F1 and F2. Two change regions V, V1 can then be specified.

A vessel simulation can be carried out, for example, with a photon counting CT with 140 kV radiation energy. Therein the stenosis which often consists of calcium, can be readily identified even with different iodine concentrations.

FIG. 4 shows absorptions by different contrast media. Herein, the absorptions are given on the Y-axis in relation to the radiation energy on the X-axis of iodine I, gadolinium Gd, gold Au and bismuth Bi.

Finally, it should again be noted that the invention described above in detail merely involves exemplary embodiments which can be modified by a person skilled in the art in a wide variety of ways without departing from the scope of the invention. Furthermore, the use of the indefinite article “a” or “an” does not preclude the possibility that the relevant features can also be present plurally. Similarly, expressions such as “unit” do not preclude the relevant components consisting of a plurality of cooperating sub-components which can also be spatially distributed, if relevant. The expression “a number” is to be understood as meaning “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative herein descriptors used interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, diagrams, block etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program instructions, code, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, JavaR, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for supporting a determination of a vessel morphology based on spectral CT information, the method:
providing at least two 3D CT images of a person recorded with different recording energies, wherein at least one low energy image and one high energy image are provided, the high energy image recorded with a higher energy than the low energy image;
selecting a vessel of the person and an examination position of the vessel;
generating an examination image by superimposing image information of at least the low energy image and the high energy image, wherein the examination image shows a cross-section through the selected vessel at the examination position in a view from above the selected vessel;
determining a checking region in the cross-section and forming a working set from image values of the checking region;
calculating at least a first derivative of the working set with respect to locations of the image values;
establishing a number of change regions in a progression of the image values of the working set based on the first derivative; and
outputting information regarding positions of the number of change regions.

2. The method of claim 1, further comprising:
calculating image values of image elements of the examination image from at least one of a quotient or a difference of values of corresponding image elements of the low energy image and of the high energy image.

3. The method of claim 1, wherein the determining the checking region includes at least one of,
forming the working set from image values on a line transversely through the vessel cross-section, wherein the first derivative of the working set is a derivative over the progression of image values along the line,
the working set comprises image points of an area of the cross-section, wherein the first derivative of the working set are partial derivatives of the working set along two linearly independent spatial directions transversely to the cross-section, or
the working set comprises image points of a volume of the vessel from the cross-section and a predetermined stretch along the vessel, wherein the first derivative of the working set are the partial derivatives of the working set along three linearly independent spatial directions.

4. The method of claim 1, wherein change regions of the image values of the working set are established from at least one of extreme sites or an amplitude of the first derivative.

5. The method of claim 1, further comprising:
forming a second derivative from the first derivative; and
establishing change regions from the second derivative or from the first derivative and the second derivative.

6. The method of claim 1, wherein during the generating the examination image, at least one of a normalization, a calibration, an adaptation, or a mapping of values additionally takes place.

7. The method of claim 1, wherein the CT images are recorded after administration of a contrast medium.

8. The method of claim 1, wherein the CT images are recorded in a context of a photon-counting CT method.

9. The method of claim 8, wherein a spectral information item from image values of the working set is established and change regions are established based on the spectral information item.

10. An apparatus for supporting a determination of a vessel morphology based on spectral CT information, the apparatus comprising:
a data interface configured to receive image data from 3D CT images of a person, the 3D CT images recorded with different recording energies at least from a low energy image and a high energy image, the high energy image recorded with a higher energy than the low energy image;
a selecting unit configured to select a vessel of the person and an examination position of the vessel;
a superimposing unit configured to generate an examination image by superimposing image information of at least the low energy image and the high energy image, wherein the examination image shows a cross-section through the selected vessel at the examination position in a view from above the selected vessel;
a working set unit configured to determine a checking region in the cross-section and forming a working set from image values of the checking region;
a calculating unit configured to calculate at least a first derivative of the working set with respect to locations of the image values;
a change unit configured to establish a number of change regions in a progression of the image values of the working set based on this derivative; and
a data interface configured to output positions of the number of change regions.

11. The apparatus of claim 10, wherein
the superimposing unit is configured to superimpose three or more CT images to the examination image, and
the calculating unit is configured to calculate a first derivative of a spectral progression above a spectral energy and the change unit is configured to establish a change region based on the derivative of the spectral progression.

12. A control facility for a medical technology system, comprising:
the apparatus of claim 10.

13. A medical technology system comprising:
the control facility of claim 12.

14. A non-transitory computer program product comprising commands which, when executed by a computer, cause said computer to perform the method of claim 1.

15. A non-transitory computer-readable memory storage medium comprising commands which, when executed by a computer, cause said computer to perform the method of claim 1.

16. The method of claim 5, wherein zero points of the second derivative are established.

17. The method of claim 6, wherein the at least one of the normalization, the calibration, the adaptation, or the mapping of values additionally takes place by way of multiplication of all the image information by a predetermined factor or a predetermined location-dependent function across the examination image.

18. The method of claim 7, wherein the contrast medium is an iodine-containing contrast medium.

19. The method of claim 8, wherein the examination image is generated by superimposing image information of a number of additional CT images in addition to the low energy image and the high energy image.

20. The method of claim 9, wherein a progression of image values from different CT images is generated dependent upon a radiation energy and a derivative of the progression above a radiation energy is calculated.
